(19) United States
(12) Patent Application Publication
(10) Pub. No.: US 2026/0295266 A1
(43) Pub. Date: Oct. 1, 2026

(54) OPTIMIZING SENSING USING AN ACCELEROMETER

(71) Applicant: Medtronic, Inc., Minneapolis, MN (US)

(72) Inventors: Joshua O. Usoro, Minneapolis, MN (US); Leonid M. Litvak, Beit Shemesh (IL); Kristin N. Hageman, Dayton, MN (US); Robert A. Corey, Arden Hills, MN (US)

(21) Appl. No.: 19/479,378

(22) PCT Filed: Apr. 11, 2024
(86) PCT No.: PCT/IB2024/053544

§ 371 Date: Oct. 28, 2025

Related U.S. Application Data
(60) Provisional application No. 63/498,911, filed on Apr. 28, 2023.

(51) Int. Cl. A61N 1/36

(52) U.S. Cl. CPC A61N 1/36139; A61N 1/36062; A61N 1/36067; A61N 1/36185

(57) ABSTRACT

Techniques to determine a physical state of a patient and, based on the physical state, dynamically update one or more parameters that define operation of a medical device, such as, one or more stimulation parameters, one or more sense parameters, and/ or signal processing parameters (e.g., artifact cancellation parameters). These parameters may be utilized by the medical device to sense physiological signals from a patient and/or deliver electrical stimulation to target tissue of the patient. In some examples the medical device may include an implantable medical device (IMD). The physical state may include one or more of a posture state, an activity state, and in some examples, a degree of distraction of the patient. Electrode position relative to target tissue may change with physical state.

This Application claims priority from U.S. Provisional Patent Application 63/498,911, filed 28 Apr. 2023, the entire content of which is incorporated herein by reference.

### TECHNICAL FIELD

The disclosure relates to medical systems configured to sense physiological signals.

### BACKGROUND

Medical devices may be external or implanted and may be used to sense physiological signals and/or deliver electrical stimulation to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS) including tibial nerve stimulation (TNS), respectively. Electrical stimulation may be delivered by the medical device as a train of pulses, and the values of the parameters defining the pulses may be altered.

### SUMMARY

In general, the disclosure describes techniques to determine a patient state and, based on the patient state, dynamically update one or more parameters that define operation of a medical device, such as, one or more stimulation parameters, one or more sense parameters, and/or signal processing parameters (e.g., artifact cancellation parameters). These parameters may be utilized by a medical device to sense physiological signals from a patient and/or deliver electrical stimulation to target tissue of the patient. In some examples the medical device may include an implantable medical device (IMD). The patient state may include one or more of a posture state, an activity state, and in some examples, a degree of distraction of the patient.

Positional changes during neuromodulation may impact the tissue-electrode coupling, thereby affecting the efficacy of stimulation delivery, sensing of evoked responses, and/or stimulation artifact morphology as seen by the sensing electrodes. When a patient moves, the distance between implanted electrodes and target nerves can change, muscle contractions can generate bioelectrical signals, and the movement itself may generate an artifact that may be sensed by the medical device. For example, electrodes implanted along the spinal column are closer to the spinal cord when a subject lies in a supine posture state as compared to a standing posture state. Similarly, the implanted electrodes may move closer to the spinal cord when a subject coughs, sneezes, or laughs. This changing distance between the electrodes and target tissue can affect neural recruitment for a given intensity of delivered stimulation and can cause the patient's perception and/or therapeutic benefit to also change. This change in neural recruitment may also change a resulting evoked signal that can be sensed by the medical device.

Stimulation artifacts can occlude the ability to sense evoked compound action potentials (ECAPs). This disclosure includes ideas to dynamically adjust stimulation and sense parameters, as well as filtering settings and algorithm deployment (stimulation artifact) triggered by positional changes detected by accelerometers on our stimulators.

In one example, this disclosure describes a device comprising processing circuitry configured to: control stimulation generation circuitry to generate an electrical stimulation signal deliverable to a patient; receive, via first sensing circuitry, an indication of a physical state of the patient; determine the physical state of the patient based on the indication of the physical state from the first sensing circuitry; and automatically determine, based on the physical state of the patient, at least one sensing parameter that at least partially defines second sensing circuitry operation to sense an evoked response signal elicited by the stimulation generation circuitry, and control the second sensing circuitry to sense the evoked response signal according to the at least one sensing parameter.

In another example, this disclosure describes a method comprising controlling, by processing circuitry operatively coupled to a memory, stimulation generation circuitry to generate an electrical stimulation signal deliverable to a patient; receiving, by the processing circuitry and via first sensing circuitry, an indication of a physical state of the patient; determining the physical state of the patient based on the indication of the physical state from the first sensing circuitry; and automatically determining, based on the physical state of the patient, at least one sensing parameter that at least partially defines second sensing circuitry operation to sense an evoked response signal elicited by the stimulation generation circuitry, and controlling, by the processing circuitry, the second sensing circuitry to sense the evoked response signal according to the at least one sensing parameter.

In another example, this disclosure describes a non-transitory computer-readable storage medium comprising instructions that, when executed, cause processing circuitry of a computing device to: control stimulation generation circuitry to generate an electrical stimulation signal deliverable to a patient; receive via first sensing circuitry, an indication of a physical state of the patient; determine the physical state of the patient based on the indication of the physical state from the first sensing circuitry; and automatically determine, based on the physical state of the patient, at least one sensing parameter that at least partially defines second sensing circuitry operation to sense an evoked response signal elicited by the stimulation generation circuitry, and control the second sensing circuitry to sense the evoked response signal according to the at least one sensing parameter.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) configured to deliver spinal cord stimulation (SCS) therapy and an external computing device, in accordance with one or more techniques of this disclosure.

FIG. 2A is a block diagram illustrating an example combination of components of an IMD, in accordance with one or more techniques of this disclosure.

FIG. 2B is a block diagram illustrating an example combination of components of an example external computing device, in accordance with one or more techniques of this disclosure.

FIG. 3A is a conceptual diagram of a lead having segmented electrodes in according to one or more techniques of this disclosure.

FIG. 3B is a conceptual cross-sectional diagram of electrode lead.

FIG. 4 is a graph illustrating an example evoked compound action potentials (ECAPs) sensed for respective stimulation pulses, in accordance with one or more techniques of this disclosure.

FIG. 5 is a timing diagram illustrating another example of electrical stimulation pulses and respective sensed ECAPs, in accordance with one or more techniques of this disclosure.

FIG. 6 is a flowchart illustrating an example operation of a medical system for automatically adjusting sensing and other parameters according to one or more techniques of this disclosure.

FIG. 7 is a flowchart illustrating an example operation of the medical system of this disclosure including automatically adjusting parameters based on patient state.

### DETAILED DESCRIPTION

The techniques of this disclosure include determining the patient state and responsive to the patient state, dynamically and automatically changing any one or more of sense parameters, artifact stimulation parameters, or stimulation parameters. The devices, and systems, of this disclosure may sense physiological signals, and may sense signals evoked by stimulation signals, such as an ECAP and stimulation artifact. However, the appropriate sensing or stimulation delivery parameters may be different for different physical states of the patient, e.g., activity level and posture.

The medical device or system of this disclosure may use one or more sensors, such as an accelerometer, gyroscope, temperature sensor, or similar sensors to determine, for example, the physical state (e.g., posture state and activity state) of a patient. The physical state may trigger the medical device to dynamically update stimulation parameters (e.g., amplitude, waveform, electrode configuration, pulse width, and frequency), sense parameters (e.g., electrode selection, gain settings, received signal post processing, and amplifier settings), and/or as artifact cancellation parameters (e.g., filter coefficients, algorithm deployment, selection of passive or active recharge). For example, any of these parameters may be adjusted to improve sensing or stimulation performance for the particular physical state of the patient.

Changes in patient posture as well as movement, e.g., walking, golfing, or inactivity, as detected by a system may trigger a change to one or more parameters defining at least one closed loop scheme used by the system to manage symptoms for a patient. In some examples, a medical device of this disclosure may adjust an evoked response sensing modality such as by changing the sensing parameters, changing stimulation parameters, changing the manner in which a characteristic value of an artifact and/or a sensed evoked response signal is determined, changing a parameter in response to changes to the evoked response, changing a parameter in patient disease state, and/or changing an aspect of the stimulation being delivered based on the determined patient state. In this example, one or more parameters that define the evoked response sensing closed-loop modality may be changed by the system in order to improve one or more performance aspects of the system for the detected physical state of the patient.

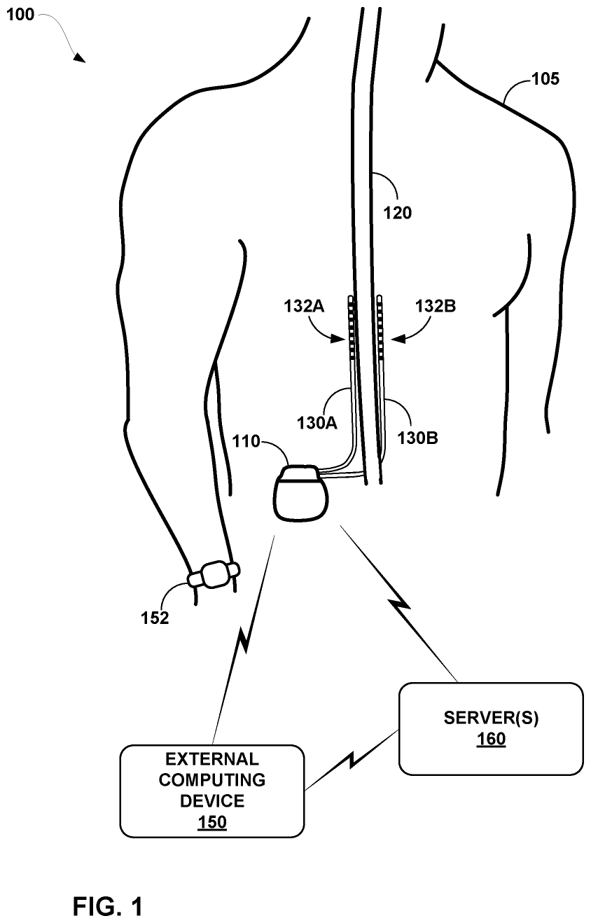

FIG. 1 is a conceptual diagram illustrating an example system 100 that includes an implantable medical device (IMD) 110 configured to deliver spinal cord stimulation (SCS) therapy and an external computing device 150, in accordance with one or more techniques of this disclosure. Although description may focus on implantable electrical stimulators (e.g., neurostimulators) for purposes of illustration, the techniques described in this disclosure are generally applicable to a variety of medical devices including external devices and IMDs. In other words, the disclosure will refer to an implantable SCS system for purposes of illustration, but without limitation as to other types of medical devices or other therapeutic applications of medical devices such as cardiac monitoring and therapy, peripheral nerve stimulation and similar devices, pelvic floor therapy, deep brain stimulation, etc.

As shown in FIG. 1, system 100 includes an IMD 110, leads 130A and 130B, and external computing device 150 shown in conjunction with a patient 105, who is ordinarily a human patient. In the example of FIG. 1, IMD 110 is an implantable electrical stimulator that is configured to generate and deliver electrical stimulation therapy to patient 105 via one or more electrodes 132A and 132B of leads 130A and/or 130B (collectively, “leads 130”), e.g., for relief of chronic pain or other symptoms. In other examples, IMD 110 may be coupled to a single lead carrying multiple electrodes, to more than two leads each carrying multiple electrodes, or without leads and where the electrodes are disposed on the housing of the IMD.

IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In some examples, IMD 110 uses one or more leads, while in other examples, IMD 110 is leadless. In other examples, IMD 110 may be located to deliver sacral neuromodulation (SNM), deep brain stimulation (DBS), and peripheral nerve stimulation (PNS), including tibial nerve stimulation (TNS).

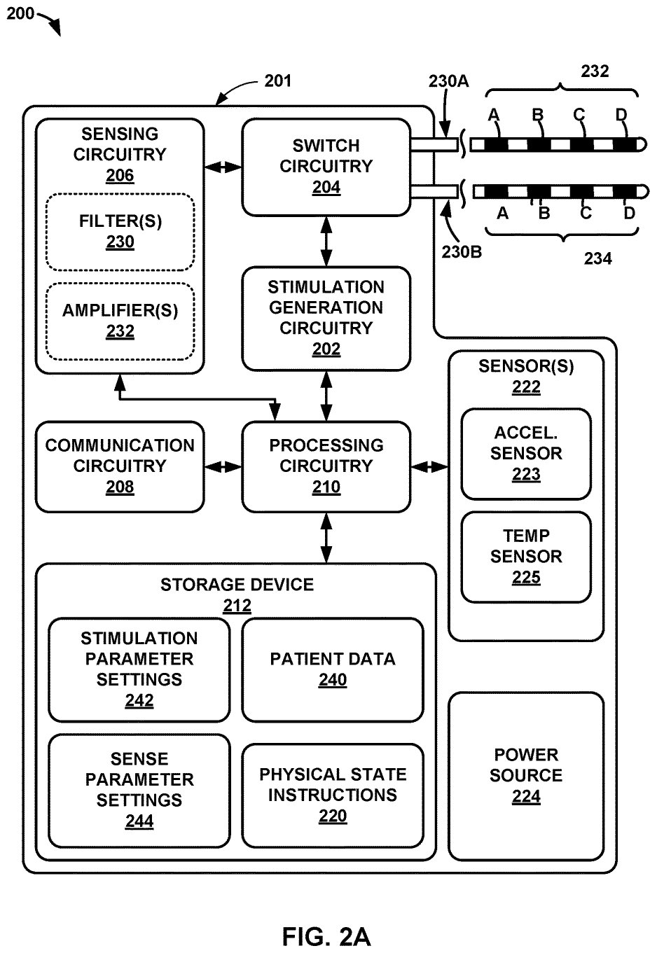

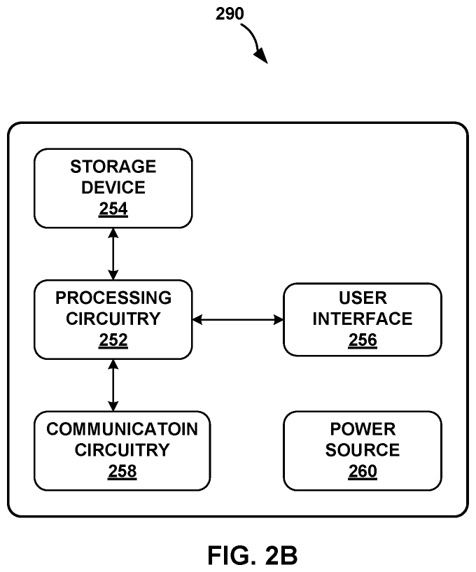

IMD 110 may be constructed of any polymer, metal, or composite material sufficient to house the components of IMD 110 (e.g., components illustrated in FIG. 2A) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted within other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source, if so equipped.

Electrical stimulation energy, which may be constant current or constant voltage-based pulses, for example, is delivered from IMD 110 to one or more target tissue sites of patient 105 via one or more electrodes (not shown) of implantable leads 130. In the example of FIG. 1, leads 130 carry electrodes that are placed adjacent to the target tissue of spinal cord 120. One or more of the electrodes 132A or 132B (collectively electrodes 132) may be disposed at a distal tip of a lead 130 and/or at other positions at intermediate points along the lead. Leads 130 may be implanted and coupled to IMD 110. Electrodes 132 may transfer electrical stimulation generated by an electrical stimulation generator (e.g., stimulation generation circuitry) in IMD 110 to tissue of patient 105.

Electrodes 132 may also sense bioelectrical signals at the electrode-tissue interface. Some examples of bioelectrical signals may include local field potentials (LFP), evoked response style sensing (e.g., evoked resonant neural activity (ERNA), evoked compound action potential (ECAP), sacral evoked responses (SER), electromyogram (EMG), compound muscle action potential (CMAP), cardiac activity, patient respiration, impedance, and similar bioelectrical signals. An ECAP is a synchronous firing of a population of neurons which occurs in response to the application of a stimulus including, in some cases, an electrical stimulus by a medical device. The ECAP may be detectable as being a separate event from the stimulus itself, and the ECAP may reveal characteristics of the effect of the stimulus on the nerve fibers. In this disclosure an ECAP may also be referred to as an electrically evoked compound action potential (EECAP).

In the example of FIG. 1, leads 130 may each be a single lead. In other examples, leads 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

Electrodes 132 of leads 130 may be electrode pads on a paddle lead, such as a 5-6-5 lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for stimulation therapy or for sensing.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having eight ring electrodes along the axial length of the lead. In another example, the electrodes are segmented rings arranged in a linear fashion along the axial length of the lead and at the periphery of the lead. Ring electrodes arranged at different axial positions at the distal ends of lead 130 in FIG. 1 will be described for purposes of illustration of the techniques of this disclosure.

In some examples, leads 130, IMD 110, or another device in communication with IMD 110 (not shown in FIG. 1) may include one or more sensors configured to allow IMD 110 to monitor one or more other parameters of patient 105, such as a physical state or other patient parameter such as pressure, temperature, or other characteristics. The one or more sensors may be in addition to, or in place of, therapy delivery by lead 130. In other examples, other sensors, such as accelerometers, gyroscopes, or other movement sensors may be configured to sense movement of the patient. Processing circuitry of IMD 110 may receive indications from such sensors to determine the movement and/or orientation of the patient which may be referred to generally as a physical state of patient 105. In this disclosure, the physical state of patient 105 may thus include a posture state and an activity state of patient 105.

As described above, different physical states of patient 105 may cause the proximity of one or more of electrodes 132 to change in relation to the target tissue of the patient. For example, changing posture from reclining to upright, or any other posture state change, may cause electrodes 132 to move relative to the target tissue. Similarly, a change in the activity state of patient 105, e.g., from stationary to walking, from asleep to awake, or to any other activity state, may also change affect the electrode tissue interface between electrodes 132 and the target tissue of patient 105. Such changes may impact sensing as well as stimulation delivery. Therefore, processing circuitry of system 100, e.g., processing circuitry of IMD 110, may receive an indication of the physical state of the patient from one or more sensors, such as an accelerometer component of IMD 110. The processing circuitry may determine the physical state of the patient based on the indication of the physical state from the first sensing circuitry. In some examples, the processing circuitry may automatically adjust, based on the physical state of the patient, at least one sensing parameter of the set of sensing parameters. In some examples, the processing circuitry may also, or alternatively, automatically adjust one or more stimulation parameters of the set of stimulation parameters, as well as other operating parameters based on the physical state of patient 105.

Some examples of sensing parameters that the processing circuity may adjust include the spacing between the sensing electrodes, and/or the orientation (e.g., the electrode combination) of at least one sensing electrode. For example, IMD 110 may change which electrodes are used as sensing electrodes to change the proximity of the sensing electrodes to the target tissue. In some examples, IMD 100 may change the orientation (e.g., which segment(s) of a segmented electrode or other electrodes are used to sense) by automatically searching the plurality of electrodes for a sensing electrode combination to determine which electrodes may be used as sensing electrodes e.g., to minimize the impact (e.g., amplitude) of a stimulation artifact or to maximize the signal strength of the sensed evoked response signal. In other examples, processing circuity of IMD 110 may also change a sensing channel, e.g., from LFP to ECAP, the amplifier gain settings, number of sensed signals to average over, or some other sensing channel adjustment based on the determined physical state.

Other sensing parameters of the set of sensing parameters, may include how the processing circuitry determines the characteristic value of the sensed evoked response signal. In some examples, processing circuitry of IMD 110 may change from determining the characteristic value based on peak-to-peak amplitude of the sensed evoked response signal to determining the characteristic value based on area under the curve. Other potential techniques for determining the characteristic value may include latency of a peak relative to an artifact, latency between peaks, the number of peaks, the frequency of the sensed evoked response signal, the slope of the artifact, decay constant of the artifact, polarity of the artifact, and/or other morphology of the artifact or the sensed evoked response signal. For example, when the characteristic value is related to latency, processing circuitry of IMD 110 may change a sensing window during which the evoked response signal is sensed, for example, by shifting the window, lengthening the window, or shortening the window. In some examples, IMD 110 may change the sensing window based on a proximity of the sensing electrodes to the target tissue based on the determined physical state of patient 105.

The set of stimulation parameters of a therapy stimulation program that defines the stimulation signal of electrical stimulation therapy by IMD 110 through electrodes 132 of leads 130 may include information identifying which electrodes have been selected for delivery of stimulation according to a stimulation program, the polarities of the selected electrodes, i.e., the electrode combination for the program, voltage or current amplitude, pulse rate (e.g., pulse frequency), pulse width, pulse shape of stimulation delivered by the electrodes, interpulse interval, a number of interleaved pulses, passive recharge settings, etc. These stimulation parameter values that make up the stimulation parameter set that defines the stimulation signal may be predetermined parameter values defined by a user and/or automatically determined by system 100 based on one or more factors or user input.

IMD 110 may configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle of the anatomy of patient 105. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical, or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may reduce the perception of pain by patient 105, and thus, provide efficacious therapy results.

A user, such as a clinician or patient 105, may interact with a user interface of an external computing device 150 to program IMD 110. Programming of IMD 110 may refer generally to the generation and transfer of commands, programs, or other information to control the operation of IMD 110. In this manner, IMD 110 may receive the transferred commands and programs from external computing device 150 to control stimulation, such as electrical stimulation therapy to develop the growth curve. For example, external computing device 150 may transmit therapy stimulation programs, evoked response stimulation programs, stimulation parameter adjustments, therapy stimulation program selections, evoked response program selections, user input, or other information to control the operation of IMD 110, e.g., by wireless communication or wired connection.

In some cases, external computing device 150 may be characterized as a physician or clinician programmer if it is primarily intended for use by a physician or clinician. In other cases, external computing device 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy, when a patient perceives stimulation being delivered or when a patient terminates due to comfort level. In general, a physician or clinician programmer may support selection and generation of programs by a clinician for use by IMD 110, whereas a patient programmer may support adjustment and selection of such programs by a patient during ordinary use.

In other examples, external computing device 150 may include, or be part of, an external charging device that recharges a power source of IMD 110. In this manner, a user may program and charge IMD 110 using one device, or multiple devices. External computing device may be any device with processing circuitry configured to communicate with other computing devices and perform calculations and other processing tasks. Some examples of external computing device 150 include wearable device 152, such as a fitness tracker, a tablet or laptop computer, a mobile phone or similar computing device (not shown in FIG. 1).

As described herein, information may be transmitted between external computing device 150 and IMD 110. Therefore, IMD 110 and external computing device 150 may communicate via wireless communication using any techniques known in the art. Examples of communication techniques may include, for example, radiofrequency (RF) communication and inductive coupling, but other techniques are also contemplated. In some examples, external computing device 150 includes a communication head that may be placed proximate to the patient's body near the IMD 110 implant site to improve the quality or security of communication, and/or power transfer, between IMD 110 and external computing device 150. Communication between external computing device 150 and IMD 110 may occur during power transmission or separate from power transmission.

In some examples, IMD 110 may also detect evoked response signals from the stimulation signal delivered to patient 105 for the purpose of modifying therapy delivered to the patient. During delivery of control stimulation pulses defined by one or more evoked response test stimulation programs, IMD 110, via two or more electrodes interposed on leads 130, senses electrical potentials of tissue of the spinal cord 120 of patient 105 to measure the electrical activity of the tissue. IMD 110 senses evoked response from the target tissue of patient 105, e.g., with sensing electrodes on one or more leads 130 and associated sensing circuitry. In some examples, IMD 110 receives a signal indicative of the evoked response from one or more sensors, e.g., one or more electrodes and circuitry, internal or external to patient 105. Such an example signal may include a signal indicating an evoked response of the tissue of patient 105.

Once the target evoked response characteristic values are set, and in addition to determining the physical state of patient 105, the example techniques allow for automatic adjustment of parameter values of the set of parameter values that define the stimulation signal to maintain consistent volume of neural activation and consistent perception of therapy for the patient. The ability to change the stimulation parameter values may also allow the therapy to have long term efficacy, with the ability to keep the intensity of the stimulation (e.g., as indicated by the evoked response) consistent by comparing the measured evoked response values to the target evoked response characteristic value. In addition, or alternatively, to maintaining stimulation intensity, IMD 110 may monitor the characteristic values of the evoked response signals to limit one or more parameter values that define the stimulation signal. IMD 110 may perform these changes without intervention by a physician or patient 105. In this manner, IMD 110 may deliver closed loop stimulation therapy.

Moreover, an IMD and/or electrodes used for stimulation and sensing may migrate within the patient, or the patient may change postures placing more or less pressure on areas containing the IMD and/or lead(s) thereby causing the electrodes, e.g., attached to the housing of IMD 110 or electrodes 132 on leads 130, to move closer or farther from the target tissue than at other times. Additionally, a patient disease state may change over time. Therefore, it may be desirable to dynamically and automatically change parameters, such as sensing parameters and/or stimulation parameters to more accurately identify the evoked response and thereby configure the electrical stimulation in a closed loop manner to deliver more efficacious therapy.

In some examples, responsive to the determined physical state of patient 105, processing circuitry of IMD 110 may adjust other operating parameters of IMD 110, in addition to, or alternative to, stimulation or sensing parameters. IMD 110 may include artifact rejection circuitry which may include one or more filters to remove or reduce the impact of any artifact in the sensed evoked response signal. In some examples, the parameters that define the operation of the artifact rejection circuitry may be referred to as part of the sensing parameters. For example, IMD 110 may adapt stimulation parameters when an artifact in the sensed evoked response signal temporally shifts within a sensing window. IMD 110 may adapt stimulation parameters based on the magnitude (e.g., amplitude or area under the curve) of the artifact, the slope of the artifact, decay constant of the artifact, polarity of the artifact, and/or other morphology of the artifact. IMD 110 may adjust filtering parameters, such as frequency levels or filtering coefficients, based on the magnitude (e.g., amplitude or area under the curve) of the artifact, the slope of the artifact, decay constant of the artifact, polarity of the artifact, and/or other morphology of the artifact. IMD 110 may also adjust filtering parameters, or other artifact rejection parameters based on the determined physical state of patient 105.

Operating parameters may also include the operation of amplification circuitry of IMD 110. IMD 110 may include amplification circuitry to amplify the sensed evoked response signal and other bioelectrical signals. IMD 110 may determine a window, having a time duration, for amplification of the sensed evoked response signal and determine blanking parameters. IMD 110 may weight temporal data within the window and/or may weight calculated features (e.g., within the morphology of the temporal data) of the temporal data within the window. In some examples, the weighting may be different for different portions of the window. For example, the weighting may be configured to emphasize the evoked response or emphasize the artifact. Processing circuitry of IMD 110 may automatically adjust the operation of the amplification circuitry based on the physical state of patient 105.

Operating parameters may also include post processing of signals received via electrodes 132, or other sensors of system 100. In some examples, IMD 110 may dynamically adjust averaging of the weighted temporal data and/or the weighting of the calculated features of the temporal data based on the physical state of patient 105 as well as based on the artifact or noise within the sensed evoked response signal. In some examples, IMD 110 may adjust the weighting of how consecutive sensed evoked response signals (and/or calculated features thereof) are averaged.

In other examples, IMD 110 may adjust a gain or other parameters of the amplification circuitry based on at least one of the calculated features or the weighted calculated features. Such calculated features may include the magnitude (e.g., amplitude or area under the curve) of the artifact, the slope of the artifact, decay constant of the artifact, polarity of the artifact, and/or other morphology of the artifact or of the sensed evoked response signal.

Other examples of sensing parameters may include a change in the polarity of the stimulation electrodes from one polarity to the opposite polarity, back and forth, and average the resulting sensed evoked response signal. In some examples, IMD 110 may also provide a masker pulse which may be delivered prior to the stimulation pulse such that the neural response to the stimulation pulse is masked due to neurons being in the refractory period due to their activation in response to the masker pulse. To facilitate this technique, the masker pulse may precede the stimulation pulse by either absolute or relative refractory period of the neural tissue (e.g., 0.3 to 5 msec). Because the neural response is masked, the recording due to pulse following the masked pulse may contain predominantly a stimulation artifact. This recording can then be used to estimate the artifact and subtract the artifact from subsequent stimulation pulses, to achieve a better estimate of underlying neural response.

In some examples, IMD 110 may also change at least one passive recharge setting of the stimulation parameters based on a time constant of the artifact. Passive recharge refers to the exponential decay of accumulated charge from a stimulation pulse through DC blocking capacitors and tissue resistance. For example, IMD 110 may change a delay, a duration, truncation, ratio, etc. of passive recharge settings. For example, IMD 110 may shorten a duration of passive recharge based on an artifact trend. In some examples, IMD 110 may change a pulse shape of the stimulation parameters in an attempt to improve the quality of the sensed evoked response signal. Passive recharge is different from active recharge. Active recharge refers to delivering a stimulation pulse of opposite polarity to substantially remove accumulated charge in the tissue from the delivered stimulation pulse. Active recharge pulses typically can restore charge neutrality faster than passive recharge, but active recharge may consume more power as a pulse is delivered instead of remaining charge passively discharged.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of DBS, PNS, PNFS, CS, TNS, pelvic floor stimulation, gastrointestinal stimulation, or any other stimulation therapy capable of treating a condition of patient 105.

Although in one example IMD 110 takes the form of an SCS device, in other examples, IMD 110 takes the form of any combination of DBS devices, peripheral nerve stimulators, implantable cardioverter defibrillators (ICDs), pacemakers, cardiac resynchronization therapy devices (CRT-Ds), left ventricular assist devices (LVADs), implantable sensors, orthopedic devices, or drug pumps, as examples. Moreover, techniques of this disclosure may be used to determine stimulation thresholds (e.g., perception thresholds and detection thresholds) associated any one of the aforementioned IMDs and then use a stimulation threshold to inform the intensity (e.g., stimulation levels) of therapy.

In the example of FIG. 1, IMD 110 is described as performing a plurality of processing and computing functions. However, external computing device 150, servers 160 or other processing circuitry of system 100 instead may perform one, several, or all of these functions. In this alternative example, IMD 110 functions to relay sensed signals to external computing device 150 for analysis, and external computing device 150 transmits instructions to IMD 110 to adjust the one or more stimulation parameters defining the electrical stimulation therapy based on analysis of the sensed signals. In some examples, processing circuitry of IMD 110, or other processing circuitry of system 100 may receive information from other devices of system 100, such as from wearable device 152 to determine the physical state of patient 105.

FIG. 2A is a block diagram illustrating an example combination of components of IMD 200, in accordance with one or more techniques of this disclosure. IMD 200 may be an example of IMD 110 of FIG. 1. In the example shown in FIG. 2A, IMD 200 includes stimulation generation circuitry 202, switch circuitry 204, sensing circuitry 206, communication circuitry 208, processing circuitry 210, storage device 212, sensor(s) 222, and power source 224.

In the example shown in FIG. 2A, storage device 212 may store patient data 240, stimulation parameter settings 242, sense parameter settings 244 and various thresholds discussed in this disclosure in separate memories within storage device 212 or separate areas within storage device 212. Patient data 240 may include parameter values, target characteristic values, or other information specific to the patient. In some examples, stimulation parameter settings 242 may include stimulation parameter values that define different stimulation programs selectable by the clinician or patient for therapy. In this manner, each stored therapy stimulation program, or set of stimulation parameter values, of stimulation parameter settings 242 defines values for a set of electrical stimulation parameters (e.g., a stimulation parameter set), such as a stimulation electrode combination, electrode polarity, current or voltage amplitude, pulse width, pulse rate, pulse shape, duty cycle, number of interleaved pulses, passive recharge settings, etc. Storage device 212 may also store parameter settings 242 that defines values for a set of electrical stimulation parameters configured to elicit a detectable evoked response signal, such as a stimulation electrode combination, electrode polarity, current or voltage amplitude, pulse width, pulse rate, and pulse shape, as described above in relation to FIG. 1.

Examples of sense parameter settings 244 may include device settings for how the sensing circuitry senses both evoked and non-evoked physiological signals from the patient. Some examples may include electrode configuration, sensing window, gain settings, amplifier settings, the selected channel, e.g., LFP vs ECAP channel, sampling rate, frequency of ECAP sensing (e.g., which may be different when the patient is asleep vs awake). In some examples, sense parameter settings 244 may also include post processing parameters, such as for filtering, signal feature extraction (e.g., peak detection, peak latency, etc.), template subtraction, and similar settings.

Accordingly, in some examples, stimulation generation circuitry 202 generates electrical stimulation signals in accordance with the set of electrical stimulation parameters noted above. Other ranges of stimulation parameter values may also be useful and may depend on the target stimulation site within patient 105. Stimulation signals may be of any form, such as continuous-time signals (e.g., sine waves) or the like.

Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232, 234, or directed sensed signals from one or more of electrodes 232, 234 to sensing circuitry 206. In other examples, stimulation generation circuitry 202 and/or sensing circuitry 206 may include sensing circuitry to direct signals to and/or from one or more of electrodes 232, 234, which may or may not also include switch circuitry 204. Though shown as attached to leads 230, any of electrodes 232 or 234 may be part of housing 201 or attached to housing 201 of IMD 200.

Sensing circuitry 206 is configured to monitor signals from any combination of electrodes 232, 234 according to the set of sensing parameters 244. As described above in relation to FIG. 1, based on the determined the physical state of the patient state, processing circuitry 210 may be configured to automatically cause switch circuitry 204 and sensing circuitry 206 to change the sensing electrodes used for sensing such as the spacing between the sensing electrodes, and/or the orientation (e.g., the electrode combination) of at least one sensing electrode. For example, switch circuitry 204 and sensing circuitry 206 may be configured to sense one or more bioelectrical signals via electrodes A and B of electrodes 232 and electrode C of electrodes 244. Based on a detected change of patient state, e.g., from reclining to upright and moving, processing circuitry 210 may cause switch circuitry 204 and sensing circuitry 206 to change the spacing of the electrodes such that sensing circuitry 206 senses the bioelectrical signals via electrodes A and D of electrodes 232.

In some examples, sensing circuitry 206 includes one or more amplifiers, filters, and analog-to-digital converters. For example, amplifier(s) 232 of sensing circuitry 206 may amplify a sensed response signal and/or filter(s) 230 of sensing circuitry 206 may filter a sensed response signal which may be used to remove or reduce the impact of artifacts on a sensed response signal. In some examples, a sensed response signal may include a sensed evoked response signal, such as an ECAP, SER or similar evoked response. In other examples, a response signal may include signals that are not evoked response signals, such as LFP, ECG or similar signals that in which characteristics of the sensed signal may be changed as a result of stimulation. Sensing circuitry 206 may be used to sense physiological signals, such as response signals and the sensing artifact. In some examples, sensing circuitry 206 detects evoked response from a particular combination of electrodes 232, 234. In some cases, the particular combination of electrodes for sensing the response includes different electrodes than a set of electrodes 232, 234 used to deliver stimulation pulses. Alternatively, in other cases, the particular combination of electrodes used for sensing response includes at least one of the same electrodes as a set of electrodes used to deliver stimulation pulses to patient 105. Sensing circuitry 206 may provide signals to an analog-to-digital converter (not shown in FIG. 2A), for conversion into a digital signal for processing, analysis, storage, or output by processing circuitry 210. As described above, and in relation to FIG. 1, processing circuitry may automatically change a sensing parameter of the set of sensing parameters based on, and in response to, the determined patient state. In some examples, changes to the patient state sensed by processing circuitry 210 may result in processing circuitry 210 changing sensing parameters for signals that are not evoked, and may not be impacted by some stimulation. For an ECG in some examples may be unaffected by some stimulation applied to, for example, sacral nerve tissue or tibial nerve tissue.

Communication circuitry 208 supports wireless communication between IMD 200 and an external programmer (not shown in FIG. 2A) or another computing device e.g., as shown in system 100 of FIG. 1. Processing circuitry 210 of IMD 200 may receive, as updates to programs, values for various stimulation parameters such as amplitude and electrode combination, from the external programmer via communication circuitry 208. Processing circuitry 210 may store updates to the stimulation parameter settings 242 or any other data in storage device 212. Communication circuitry 208 in IMD 200, as well as communication circuitry in other devices and systems described herein, such as the external computing device, may accomplish communication by radiofrequency (RF) communication techniques. In addition, communication circuitry 208 may communicate with an external medical device programmer (not shown in FIG. 2A) via proximal inductive interaction of IMD 200 with an external computing device.

The external computing device may be one example of external computing device 150 of FIG. 1. Accordingly, communication circuitry 208 may send information to the external computing device on a continuous basis, at periodic intervals, or upon request from IMD 110 or the external computing device. As described above in relation to FIG. 1, processing circuitry of system 100 may receive information from IMD 200 and may perform any of the analysis described herein. For example, external computing device 150 may determine that the patient physical state has changed from a first physical state to a second physical state. The processing circuitry may automatically adjust at least one stimulation parameter of the set of stimulation parameters based on both the sensed evoked response signal and the determined physical state of the patient. If external to IMD 200, the external processing circuitry may communicate with processing circuitry 210 to control delivery of the electrical stimulation according to the adjusted value of the at least one stimulation parameter. Similarly, processing circuitry of system 100, such as processing circuity of IMD 110, may automatically adjust at least one sensing parameter of the set of sensing parameters including artifact cancellation parameters, based on determining a change in patient physical state. In some examples, system 100 may determine the physical state of the patient by selecting one physical state of a plurality of potential physical states for the patient. System 100 may compare characteristics of one or more sensed signals to aspects of the different physical states in order to select the physical state of the patient.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software, or any combination thereof. Processing circuitry 210 controls stimulation generation circuitry 202 to generate stimulation signals according to stimulation parameter settings 242 and any other instructions stored in storage device 212 to apply stimulation parameter values specified by one or more of programs, such as electrode combination, electrode polarity, amplitude, pulse width, pulse rate, pulse shape, number of interleaved pulses, passive recharge settings, etc., of each of the stimulation signals.

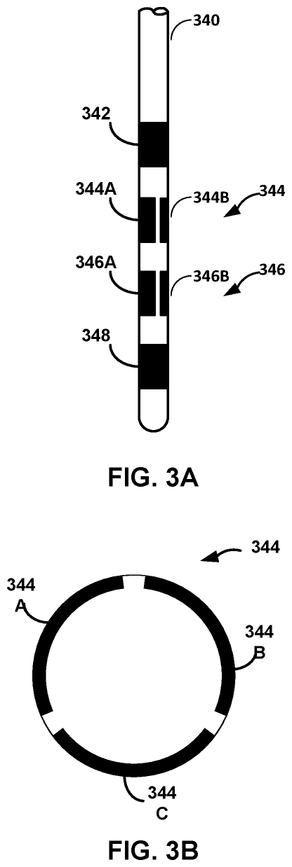

In the example shown in FIG. 2A, lead 230A is shown having a set of electrodes 232 that includes electrodes 232A-232D, and lead 230B is shown having a set of electrodes 234 includes electrodes 234A-234D. However, lead 230A and lead 230B may have any number of electrodes, such as 8 electrodes or 16 electrodes. In other examples, a single lead may be coupled to IMD 200 which may include any number of electrodes, such as include 8 electrodes or 16 electrodes along a single axial length of the lead. In some examples, one or more leads may include electrodes as shown in FIGS. 3A and 3B. In other examples, as described above in relation to FIG. 1, IMD 200 may include electrodes on the housing of IMD 200 and in some examples be configured as a leadless device (not shown in FIG. 2A)

Processing circuitry 210 also controls stimulation generation circuitry 202 to generate and apply the stimulation signals to selected combinations of electrodes 232, 234. In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord of the patient (not shown in FIG. 2A) with selected electrodes 232, 234. As described above in relation to FIG. 1, electrodes 232 and 234 may also be configured to sense other bioelectrical signals.

In other examples, however, stimulation generation circuitry 202 does not include a switch circuit and switch circuitry 204 does not interface between stimulation generation circuitry 202 and electrodes 232, 234. In these examples, stimulation generation circuitry 202 includes a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 202, e.g., via switch circuitry 204 and/or switching circuitry of the stimulation generation circuitry 202, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 206 is incorporated into a common housing with stimulation generation circuitry 202 and processing circuitry 210 in FIG. 2A, in other examples, sensing circuitry 206 may be in a separate housing from IMD 200 and may communicate with processing circuitry 210 via wired or wireless communication techniques. In some examples, one or more of electrodes 232 and 234 are suitable for sensing the evoked responses. For instance, electrodes 232 and 234 may sense the voltage amplitude of a portion of the evoked response signals, where the sensed voltage amplitude, such as the voltage difference between features within the signal, is a characteristic the evoked response signal.

Processing circuitry 210 may automatically adapt stimulation parameters when an artifact in the sensed evoked response signal shifts in a sensing window. Processing circuitry 210 may determine that at least one of the artifact or the sensed evoked response signal is more and a difference threshold different than an expected evoked response signal and refrain from changing the stimulation parameters based on the sensed evoked response signal (e.g., temporarily stopping the use of closed-loop stimulation, in which case stimulation generation circuitry 202 may continue to deliver stimulation using the existing stimulation parameters). In some examples, processing circuitry 210 may automatically suspend closed-loop stimulation based on a determined patient physical state. In other words, processing circuitry 210 may be configured to change operation of the device based on the determined patient state. For example processing circuitry 210 may cause stimulation circuitry 202 to suspend delivery of the stimulation signal if IMD 200 is delivering stimulation or deliver the stimulation signal, e.g., restart stimulation, if the stimulation signal is suspended.

In some examples, processing circuitry 210 may automatically adjust post processing techniques in response to determining a patient physical state. Processing circuitry 210 may determine a window, e.g., having a time duration, for amplification of the sensed evoked response signal for amplifier(s) 232. Processing circuitry 210 may weight temporal data within the window. In some examples, the weighting may be different for different portions of the window. By weighting an area of the window having the artifact with a lower weight than the other areas of the window, processing circuitry 210 may reduce or remove the artifact from the sensed evoked response signal. Processing circuitry 210 may adjust averaging of the weighted temporal data based on noise within the sensed evoked response signal.

In some examples, processing circuitry 210 may change stimulation parameters in an attempt to improve the quality of the sensed evoked response signal. For example, processing circuitry 210 may, in response to determining the patient physical state, alternate the polarity of the stimulation electrodes and average the sensed evoked response signal over time. For example, processing circuitry 210 may change the polarity of the stimulation electrodes from one polarity to the opposite polarity, back and forth, and average the resulting sensed evoked response signal. For example, if the stimulation electrodes are electrodes 232D and 234D and electrode 232D functions as an anode and electrode 234D functions as a cathode, processing circuitry 210 may change electrode 232D to function as the cathode and electrode 234D to function as the anode. In other examples, processing circuitry 210 may control stimulation generation circuitry 202 to deliver a probe pulse and a mask the neural response (thereby unmasking the artifact). Processing circuitry 210 may then subtract the captured artifact waveform from subsequent sensed evoked response signals without the masked pulse to identify the evoked response in the sensed evoked response signal. In some examples, the masker pulse may be the opposite polarity or differ in amplitude from the probe pulse.

In other examples, processing circuitry 210 may retrieve a stored template of the artifact, or of some other signal to be suppressed. The retrieved template may be one of several templates stored at storage device 212. In some examples the stored templates may each be based on different postures or movements. Processing circuitry 210 may retrieved the stored template, e.g., of the artifact, based on the determined patient state, then subtract the stored template from the signal received by sensing circuitry 206 to suppress the undesired signal.

In some examples, processing circuitry 210 may determine, based on the determined patient state, and measurements of the bioelectrical signals, that the amplitude of the artifact may have grown. In response to that determination, processing circuitry 210, or other processing circuitry in system 100 depicted in FIG. 1, may change a number of interleaved pulses of the stimulation parameters. For example, if the number of interleaved pulses was previously 10, processing circuitry 210 may increase or decrease that number in an attempt to improve the signal quality of the sensed evoked response signal. In some examples, processing circuitry 210 may change at least one passive recharge setting of the stimulation parameters based on a time constant of the artifact. In other examples, active recharge setting adjustments may include spacing or timing the recharge pulse, amplitude of the recharge pulse (higher amp and shorter pulse width, but overall same charge) or partially passive and partially active recharge to remove charge. In some examples, processing circuitry 210 may change a pulse shape of the stimulation parameters in an attempt to improve the quality of the sensed evoked response signal.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), ferroelectric random access memories (FRAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210. As discussed above, storage device 212 is configured to store patient data 240, stimulation parameter 214, and sense parameter settings 244.

In some examples, storage device 212 may store evoked response detection instructions which may include instructions on how processing circuitry 210 can adjust stimulation (e.g., stimulation parameter settings 242), the manner in which evoked responses are sensed, and/or the manner in which characteristic values are determined, in response to the determined characteristic values of evoked response signals or artifacts.

As described above in relation to FIG. 1, sensor(s) 222 may include one or more sensing elements that sense values of a respective patient parameter, such as posture state and activity state. Sensor(s) 222 may include one or more accelerometers, and gyroscopes, such as a micro electro-mechanical system (MEMS) based device. Other sensors may include optical sensors, chemical sensors, temperature sensors, pressure sensors, or any other types of sensors. Sensor(s) 222 may output patient parameter values that may be used as feedback to control delivery of therapy. For example, sensor(s) 222 may indicate physical state, including patient activity, and processing circuitry 210 may increase the frequency of control pulses and evoked response sensing in response to detecting increased patient activity.

In some examples, processing circuitry 210 may initiate control pulses and corresponding evoked response sensing in response to a signal from sensor(s) 222 indicating that patient activity has exceeded an activity threshold. Conversely, processing circuitry 210 may decrease the frequency of control pulses and evoked response sensing in response to detecting decreased patient activity. For example, in response to sensor(s) 222 no longer indicating that the sensed patient activity exceeds a threshold, processing circuitry 210 may suspend or stop delivery of control pulses and evoked response sensing. In this manner, processing circuitry 210 may dynamically deliver control pulses and sense evoked response signals based on patient activity to reduce power consumption of the system when the electrode-to-neuron distance is not likely to change and increase system response to evoked response changes when electrode-to-neuron distance is likely to change. IMD 200 may include additional sensors within the housing of IMD 200 and/or coupled via one of leads 230 or other leads. In addition, IMD 200 may receive sensor signals wirelessly from remote sensors via communication circuitry 208, for example. In some examples, one or more of these remote sensors may be external to patient (e.g., carried on the external surface of the skin, attached to clothing, or otherwise positioned external to patient 105, such as wearable device 152 shown in FIG. 1). In some examples, signals from sensor(s) 222 indicate the physical state including a position, body state, or posture (e.g., sleeping, awake, sitting, standing, or the like), and processing circuitry 210 may select target evoked response characteristic values according to the indicated physical state of the patient.

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. Power source 224 may include any one or more of a plurality of different battery types, such as nickel cadmium batteries and lithium ion batteries.

FIG. 2B is a block diagram illustrating an example combination of components of an example external computing device 290. External computing device 290 may be an example of external computing device 150, servers 160, and wearable device 152 of FIG. 1. Although external computing device 290 may generally be described as a hand-held device, external computing device 290 may be a larger portable device or a more stationary device. In addition, in other examples, external computing device 290 may be included as part of an external charging device or include the functionality of an external charging device. As illustrated in FIG. 2B, external computing device 290 may include processing circuitry 252, storage device 254, user interface 256, communication circuitry 258, and power source 260. Storage device 254 may store instructions that, when executed by processing circuitry 252, cause processing circuitry 252 and external computing device 290 to provide the functionality ascribed to external computing device 290 throughout this disclosure. Each of these components, circuitry, or modules, may include electrical circuitry that is configured to perform some, or all of the functionality described herein. For example, processing circuitry 252 may include processing circuitry configured to perform the processes discussed with respect to processing circuitry 252.

In general, external computing device 290 includes any suitable arrangement of hardware, alone or in combination with software and/or firmware, to perform the techniques attributed to external computing device 290, and processing circuitry 352, user interface 356, and communication circuitry 358 of external computing device 290. In various examples, external computing device 290 may include one or more processors, such as one or more microprocessors, DSPs, ASICs, FPGAs, or any other equivalent integrated or discrete logic circuitry, as well as any combinations of such components.

External computing device 290 also, in various examples, may include a storage device 254, such as RAM, ROM, PROM, EPROM, EEPROM, flash memory, a hard disk, a CD-ROM, including executable instructions for causing the one or more processors to perform the actions attributed to them. Moreover, although processing circuitry 252 and communication circuitry 258 are described as separate modules, in some examples, processing circuitry 252 and communication circuitry 258 are functionally integrated. In some examples, processing circuitry 252 and communication circuitry 258 correspond to individual hardware units, such as ASICs, DSPs, FPGAs, or other hardware units.

Storage device 254 (e.g., a storage device) may store instructions that, when executed by processing circuitry 252, cause processing circuitry 252 and external computing device 290 to provide the functionality ascribed to external computing device 290 throughout this disclosure. For example, storage device 254 may include instructions that cause processing circuitry 252 to obtain a parameter set from memory, select a spatial electrode pattern, or receive a user input and send a corresponding command to IMD 200, or instructions for any other functionality. In addition, storage device 254 may include a plurality of programs, where each program includes a stimulation parameter set that defines therapy stimulation or control stimulation. Storage device 254 may also store data received from a medical device (e.g., IMD 110). For example, storage device 254 may store evoked response related data sensed by sensing circuitry of the medical device, and storage device 254 may also store data from one or more sensors of the medical device.

User interface 256 may include a button or keypad, lights, a speaker for voice commands, a display, such as a liquid crystal (LCD), light-emitting diode (LED), or organic light-emitting diode (OLED). In some examples the display includes a touch screen. User interface 256 may be configured to display any information related to the delivery of electrical stimulation, identified posture states, sensed patient parameter values, or any other such information. User interface 256 may also receive user input (e.g., indication of when the patient perceives a stimulation pulse) via user interface 256. The input may be, for example, in the form of pressing a button on a keypad or selecting an icon from a touch screen. The input may request starting or stopping electrical stimulation, the input may request different stimulation parameters (e.g., to change a stimulation program), or the input may request some other change to the delivery of electrical stimulation or sensing.

Communication circuitry 258 may support wireless communication between the medical device and external computing device 290 under the control of processing circuitry 252. Communication circuitry 258 may also be configured to communicate with another computing device via wireless communication techniques, or direct communication through a wired connection. In some examples, communication circuitry 258 provides wireless communication via an RF or proximal inductive medium. In some examples, communication circuitry 258 includes an antenna, which may take on a variety of forms, such as an internal or external antenna.

Examples of local wireless communication techniques that may be employed to facilitate communication between external computing device 290 and IMD 110 include RF communication according to the 802.11 or Bluetooth® specification sets or other standard or proprietary communication protocols. In this manner, other external devices may be capable of communicating with external computing device 290 without needing to establish a secure wireless connection. As described herein, communication circuitry 258 may be configured to transmit a spatial electrode movement pattern or other stimulation parameter values to IMD 110 for delivery of electrical stimulation therapy. Although IMD 110 may determine characteristic values for evoked response signals and control the adjustment of stimulation parameter values or sensing parameters, in some examples, programmer 290 may perform these tasks alone or together with IMD 110 in a distributed function.

In some examples, selection of stimulation parameters or therapy stimulation programs and/or sensing parameters are transmitted to the medical device for delivery of stimulation to a patient (e.g., patient 105 of FIG. 1) and sensing of evoked response signals. In other examples, the therapy may include medication, activities, or other instructions that patient 105 must perform themselves or a caregiver perform for patient 105. In some examples, external computing device 290 provides visual, audible, and/or tactile notifications that indicate there are new instructions. External computing device 290 requires receiving user input acknowledging that the instructions have been completed in some examples.

User interface 256 of external computing device 290 may also be configured to receive an indication from a clinician instructing a processor of the medical device to update one or more therapy stimulation programs or to update the target characteristic values for evoked response signals. Updating therapy stimulation programs and target characteristic values may include changing one or more parameters of the stimulation pulses delivered by the medical device according to the programs, such as amplitude, pulse width, frequency, pulse shape of the pulses and/or control pulses, electrode combinations, electrode polarity, number of interleaved pulses, passive recharge settings, etc. User interface 256 may also receive instructions from the clinician commanding any electrical stimulation, including therapy stimulation and control stimulation to commence or to cease.

Power source 260 is configured to deliver operating power to the components of external computing device 290. Power source 260 may include a battery and a power generation circuit to produce the operating power. In some examples, the battery is rechargeable to allow extended operation. Recharging may be accomplished by electrically coupling power source 260 to a cradle or plug that is connected to an alternating current (AC) outlet. In addition, recharging may be accomplished through proximal inductive interaction between an external charger and an inductive charging coil within external computing device 290. In other examples, traditional batteries (e.g., nickel cadmium or lithium ion batteries) may be used. In addition, external computing device 290 may be directly coupled to an alternating current outlet to operate.

The architecture of external computing device 290 illustrated in FIG. 2B is shown as an example. The techniques as set forth in this disclosure may be implemented in the example external computing device 290 of FIG. 2B, as well as other types of systems not described specifically herein. Nothing in this disclosure should be construed so as to limit the techniques of this disclosure to the example architecture illustrated by FIG. 2B.

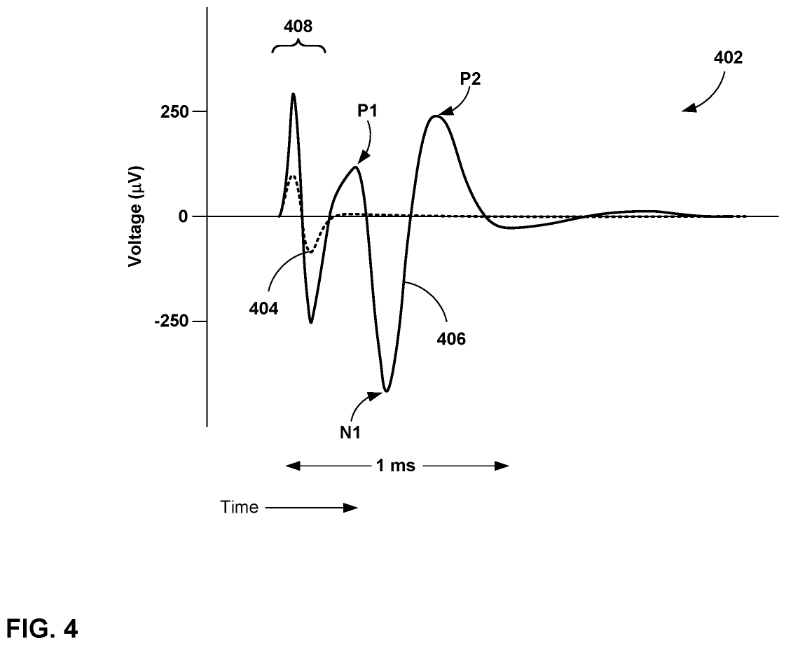

FIG. 3A is a conceptual diagram of a lead having segmented electrodes. As shown in FIG. 3A, lead 340 includes four axial electrode levels, electrode level 342, electrode level 344 (which may include segments 344A, 344B, and 344C (not shown because it may be on the far side of lead 340)), electrode level 346 (which may include segments 344B, and 344C (not shown because it may be on the far side of lead 340)) positioned at various lengths along a body of lead 340. In some examples, electrode levels 342, 344, 346C, and 348 may be equally spaced along the axial length of lead 340 at different axial positions. Each electrode level 404A-404D may have one, two, three, or more electrodes located at different angular positions around the circumference (e.g., around the perimeter) of lead 340. As shown in FIG. 4A, electrode level 342 and 348 include a single respective ring electrode, and electrode levels 344 and 346 each include three segmented electrodes at different circumferential positions. This electrode pattern may be referred to as a 1-3-3-1 lead in reference to the number of electrodes from the proximal end to the distal end of lead 340. Electrodes of one circumferential location may be lined up on an axis parallel to the longitudinal axis of lead 340. Alternatively, electrodes of different electrode levels may be staggered around the circumference of lead 340. In addition, lead 340 may include asymmetrical electrode locations around the circumference, or perimeter, of each lead or electrodes of the same level that have different sizes. These electrodes may include semi-circular electrodes that may or may not be circumferentially aligned between electrode levels.

FIG. 3B is a conceptual cross-sectional diagram of electrode lead 344 of FIG. 3A. As described above in relation to FIG. 1, processing circuitry of IMD 110 may automatically change an orientation of at least one sensing electrode based on a sensed physical state of patient 105. In some examples, based on received sensor signals, e.g., from sensors 222 in FIG. 2A, the processing circuitry may determine that the physical state of the patient is any one of an activity state such as walking, stationary, mobile, at rest, a fall, exercise, and asleep and similar physical states.

The orientation may refer to the direction that a sensing electrode may be facing or a combination of electrodes may be facing (e.g., the electrode combination used for sensing). Changing an orientation of a sensing electrode may include selecting a different sensing electrode or combination of electrodes rather than physically moving a sensing electrode. For example, IMD 110 may change a sensing electrode from electrode 344A to electrode 344B or to a combination of electrodes such as from electrode 344A to electrodes 344A and 344B or to electrodes 344B and 344C to change an orientation of a sensing electrode. In this manner, IMD 110 may change an orientation of at least one sensing electrode.

FIG. 4 is a graph 402 of example evoked compound action potentials (ECAPs) sensed for respective stimulation pulses, in accordance with one or more techniques of this disclosure. As shown in FIG. 4, graph 402 shows example ECAP signal 404 (dotted line) and ECAP signal 406 (solid line). Sensing circuitry, such as sensing circuitry 206 of FIG. 2A, may detect peaks 408 of ECAP signal 404 represent the artifact of the delivered stimulation pulse (e.g., a control pulse that may or may not contribute to a therapeutic effect for the patient). However, in some examples, the sensing circuitry may not detect the propagating signal after the artifact in ECAP signal 404 because the control pulse was sub-detection threshold.

In contrast to ECAP signal 404, ECAP signal 406 may represents the voltage amplitude detected from a supra-detection threshold control pulse. The sensing circuitry may detect peaks 408 of ECAP signal 406 and represent the artifact of the delivered control pulse. After peaks 408, ECAP signal 406 also includes peaks P1, N1, and P2, which in some examples may be three peaks representative of propagating action potentials from an ECAP. The example duration of the artifact and peaks P1, N1, and P2 may be approximately 1 millisecond (ms) in some examples.

When detecting the ECAP of ECAP signal 406, different characteristics may be identified. For example, the characteristic of the ECAP may be the amplitude between N1 and P2. This N1-P2 amplitude may be detectable even if the artifact impinges on Pl, a relatively large signal, and the N1-P2 amplitude may be minimally affected by electronic drift in the signal. In other examples, the characteristic of the ECAP may be an amplitude of P1, N1, or P2 with respect to neutral or zero voltage. In some examples, the characteristic of the ECAP may be a sum of two or more of peaks P1, N1, or P2. In other examples, the characteristic of ECAP signal 406 may be the area under one or more of peaks P1, N1, and/or P2. In other examples, the characteristic of the ECAP may be a ratio of one of peaks P1, N1, or P2 to another one of the peaks.

In some examples, the characteristic of the ECAP is a slope between two points in the ECAP signal, such as the slope between N1 and P2. In other examples, the characteristic of the ECAP may be the time between two points of the ECAP, such as the time between N1 and P2. The time between two points in the ECAP signal may be referred to as a latency of the ECAP and may indicate the types of fibers being captured by the control pulse. ECAP signals with lower latency (i.e., smaller latency values) indicate a higher percentage of nerve fibers that have faster propagation of signals, whereas ECAP signals with higher latency (i.e., larger latency values) indicate a higher percentage of nerve fibers that have slower propagation of signals. Other characteristics of the ECAP signal may be used in other examples. In some examples, the processing circuitry of the system of this disclosure may automatically adjust which characteristic of the measured bioelectrical signal to use based on the detected physical state of the patient.

In some examples, the amplitude of the ECAP signal generally increases with increased amplitude of the control pulse, as long as the pulse amplitude is greater than threshold such that nerves depolarize and propagate the signal. As discussed herein, the relationship between ECAP signal amplitude and pulse amplitude also depends on the posture state of the patient, so the relationship between the ECAP signal and pulse amplitude changes for different posture states. The target ECAP characteristic (e.g., the target ECAP amplitude) may be determined from the ECAP signal detected from a control pulse when informed pulses are determined to deliver effective therapy to patient 105. The ECAP signal thus is representative of the distance between the stimulation electrodes and the nerves appropriate for the stimulation parameter values of the informed pulses delivered at that time. IMD 110 may use the detected posture state from an ECAP characteristic value and detected changes to the measured ECAP characteristic value to change informed pulse parameter values and maintain the target ECAP characteristic value during informed pulse delivery.

In other examples, stimulation of sacral nerves through electrical leads implanted near sacral nerves via sacral neuromodulation may evoke a neural response in adjacent nerves, muscle contractions within the pelvic floor, and distal contractions in the foot. Captured stimulation-evoked signals for electrodes placed to stimulate sacral nerves may be a composite of multiple signals evoked by multiple signal sources (e.g., nerves and/or muscles) in response to delivery of electrical stimulation therapy. Such captured responses may be referred to as sacral evoked response (SER). Different signal sources may have different response times and different shapes, or morphology. For example, neural responses may differ from sensed muscle contractions, and the different sources may be located at different distances from both the electrical stimulation source (e.g., an electrode of a lead) and a sensor (e.g., which may be the same and/or a different electrode on the same and/or different lead, or a different sensor located within and/or external to the patient's body). Similar to the ECAP described above, IMD 110 may use the detected posture state from an SER characteristic value and detected changes to the measured SER characteristic value to change informed pulse parameter values and/or maintain the target characteristic values.

In other examples, other biopotentials may also present and sensed by sensing circuitry 206, such as LFPs. In some examples, LFPs may be sensed by electrodes placed in the patient's brain, as well as near the spine. Spinal LFPs may be alternately described as electrospinograms. LFPs are rhythmic oscillations that may be detectable in the brain, and from the spine and related structures in the epidural space, such as the dorsal root ganglion (DRG), dorsal roots/rootlets, the dorsal root entry zone, and ventral roots/rootlets. When recorded with electrodes 232 and 234 of leads 230 (e.g., macro-electrodes), the spontaneous activity can show up as an increase in noise levels, as well as possibly a peak in particular frequency. The characteristics of LFP's may be indicative of various disease or neurophysiologic conditions, and the LFPs may be influenced via neuromodulation, e.g., as an evoked response. LFPs may be classified by measuring the power of the LFP in one or more spectral bands. In addition to use in local closed loop control, in some instances, LFPs may also be sensed at one location to better inform, in part, neuromodulation therapy delivered at another location.

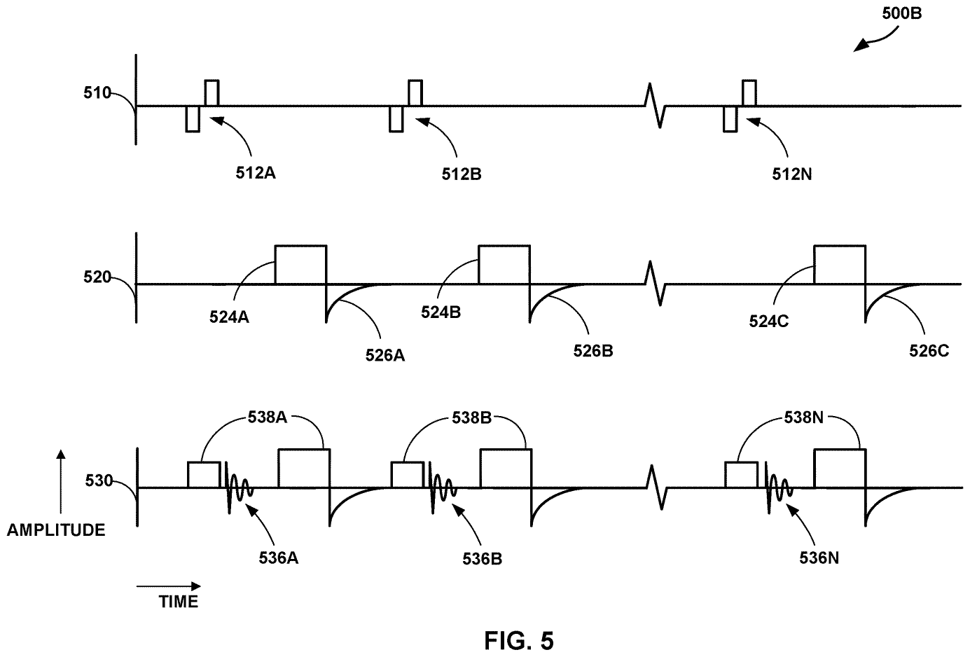

FIG. 5 is a timing diagram 500 illustrating another example of electrical stimulation pulses and respective sensed ECAPs, in accordance with one or more techniques of this disclosure. For convenience, FIG. 5 is described with reference to IMD 200 of FIG. 2. As illustrated, timing diagram 500 includes first channel 510, a plurality of control pulses 512A-512N (collectively “control pulses 512”), second channel 520, a plurality of informed pulses 524A-524N (collectively “informed pulses 524”) including passive recharge phases 526A-526N (collectively “passive recharge phases 526”), third channel 530, a plurality of respective ECAPs 536A-536N (collectively “ECAPs 536”), and a plurality of stimulation interference signals 538A-538N (collectively “stimulation interference signals 538”).

First channel 510 is a time/voltage (and/or current) graph indicating the voltage (or current) of at least one electrode of electrodes 232, 234. In one example, the stimulation electrodes of first channel 510 may be located on the opposite side of the lead as the sensing electrodes of third channel 530. Control pulses 512 may be electrical pulses delivered to the target tissue of the patient, such as the spinal cord, by at least one of electrodes 232, 234, and control pulses 512 may be balanced biphasic square pulses with an interphase interval. In other words, each of control pulses 512 are shown with a negative phase and a positive phase separated by an interphase interval. For example, control pulse 512 may have a negative voltage for the same amount of time and amplitude that it has a positive voltage. It is noted that the negative voltage phase may be before or after the positive voltage phase. Control pulses 512 may be delivered according to the set of stimulation parameters stored in memory 216 of IMD 200. The stimulation parameters may be updated according to user input via an external programmer and/or may be updated according to the determined physical state based on a signal from sensor(s) 222. As illustrated in FIG. 5, control pulses 512 may be delivered via channel 510.

Second channel 520 is a time/voltage (and/or current) graph indicating the voltage (or current) of at least one electrode of electrodes 232, 234 for the informed pulses. In one example, the electrodes of second channel 520 may partially or fully share common electrodes with the electrodes of first channel 510 and third channel 530. Informed pulses 524 may also be delivered by the same leads 230 that are configured to deliver control pulses 512. Informed pulses 524 may be interleaved with control pulses 512, such that the two types of pulses are not delivered during overlapping periods of time. However, informed pulses 524 may or may not be delivered by exactly the same electrodes that deliver control pulses 512.

Informed pulses 524 may be configured for passive recharge. For example, each informed pulse 524 may be followed by a passive recharge phase 526 to equalize charge on the stimulation electrodes. Unlike a pulse configured for active recharge, wherein remaining charge on the tissue following a stimulation pulse is instantly removed from the tissue by an opposite applied charge, passive recharge allows tissue to naturally discharge to some reference voltage (e.g., ground or a rail voltage) following the termination of informed pulse 524. In some examples, the electrodes of the medical device may be grounded at the medical device body. In this case, following the termination of informed pulse 524, the charge on the tissue surrounding the electrodes may dissipate to the medical device, creating a rapid decay of the remaining charge at the tissue following the termination of the pulse. This rapid decay is illustrated in passive recharge phases 526. Passive recharge phase 526 may have a duration in addition to the pulse width of the preceding informed pulse 524. In other examples (not pictured in FIG. 5), informed pulses 524 may be bi-phasic pulses having a positive and negative phase (and, in some examples, an interphase interval between each phase) which may be referred to as pulses including active recharge. Informed pulse 524 that is a bi-phasic pulse may or may not have a following passive recharge phase. As described above in relation to FIGS. 1 and 2, processing circuitry 210, or other processing circuitry of system 100, may automatically cause the IMD to switch between active recharge and passive recharge based on the determination by processing circuitry of the physical state of patient 105.

Third channel 530 is a time/voltage (and/or current) graph indicating the voltage (or current) of at least one electrode of electrodes 232, 234. In one example, the electrodes of third channel 530 may be located on the opposite side of the lead as the electrodes of first channel 510. ECAPs 536 may be sensed at electrodes 232, 234 from the target tissue of the patient in response to control pulses 512. ECAPs 536 are electrical signals which may propagate along a nerve away from the origination of control pulses 512. In one example, ECAPs 536 are sensed by different electrodes than the electrodes used to deliver control pulses 512. As illustrated in FIG. 5, ECAPs 536 may be recorded on third channel 530.

Stimulation interference signals 538A, 538B, and 538N (e.g., also referred to as the stimulation artifact of the stimulation pulses in this disclosure) may be sensed by leads 230 and may be sensed during the same period of time as the delivery of control pulses 512 and informed pulses 524. Since the interference signals may have a greater amplitude and intensity than ECAPs 536, any ECAPs arriving at IMD 200 during the occurrence of stimulation interference signals 538 may not be adequately sensed by sensing circuitry 206 of IMD 200. As described above in relation to FIG. 4, ECAPs 536 may be sufficiently sensed by sensing circuitry 206 because each ECAP 536 falls after the completion of each control pulse 512 and before the delivery of the next informed pulse 524. As illustrated in FIG. 5, stimulation interference signals 538 and ECAPs 536 may be recorded on channel 530.

Based on the set of stimulation and sensing parameters, two or more control pulses 512 may be delivered during each time event (e.g., window) of a plurality of time events, and each time event represents a time between two consecutive informed pulses 524. For example, during each time event, a first control pulse may be directly followed by a first respective ECAP, and subsequent to the completion of the first respective ECAP, a second control pulse may be directly followed by a second respective ECAP. Informed pulses may commence following the second respective ECAP.

Consecutive informed pulses 524 may be delivered without intervening control pulse 512. For example, control pulses 512 may not be delivered during each time event (or window) of the plurality of time events, wherein each time event represents a time between two consecutive informed pulses 524. In any case, informed pulses 524 can be delivered according to a predetermined frequency, and control pulses 512 may be delivered at any time between the informed pulses. As described above in relation to FIGS. 1 and 2, the processing circuitry may adjust one or more parameters of the set of parameters based on determined patient physical state.

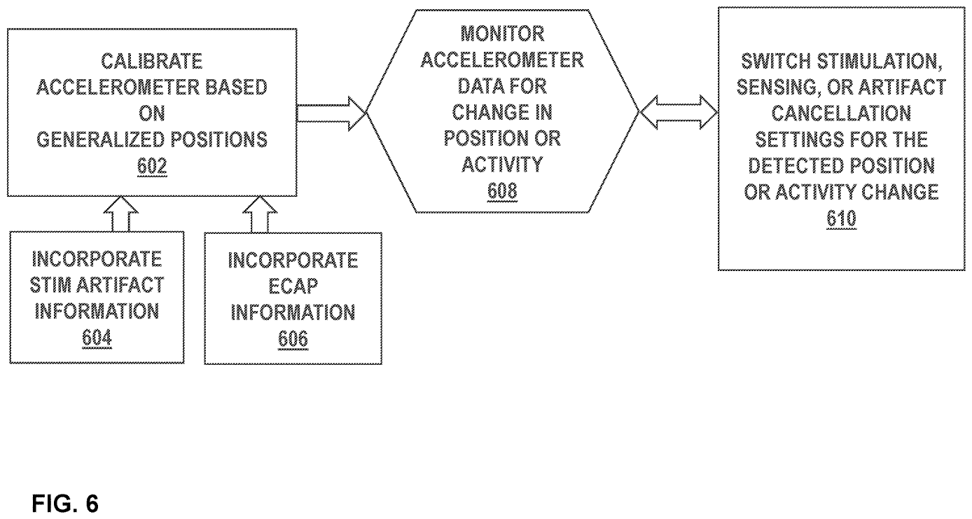

FIG. 6 is a flowchart illustrating an example operation of a medical system for automatically adjusting sensing and other parameters according to one or more techniques of this disclosure. The blocks of FIG. 6 will be described in terms of FIGS. 1 and 2 as described above.

Processing circuitry of system 100, e.g., processing circuitry 210 of IMD 200 depicted in FIG. 2, may calibrate one or more of sensors 222, such as accelerometer sensor 223, based on generalized patient physical state (602). For example, the processing circuitry may calibrate sensors 222 for a posture state of: upright, reclining, bent over, lying front, lying back, lying left, lying right and other similar generalized posture states. In some examples, reclining may also include sitting and/or leaning left or right or some similar posture. Similarly, the processing circuitry may calibrate sensors 222 for generalized activity states including walking, stationary, mobile, at rest, a fall, exercise, asleep and other activity states.

In some examples, the processing circuitry may receive and incorporate stimulation artifact information (604) and correlate with the determined physical state based on signals from sensor 222. Similarly, the processing circuitry may receive and incorporate evoked response information, such as ECAPS, (604) and correlate with the determined physical state based on signals from sensor 222. In some examples, the correlation between physical state and typical settings may be based on prior testing, e.g., clinical studies, and may also be learned over time, e.g., based on anonymous patient data from the patient population. In this manner, the system of this disclosure may help improve closed loop neuromodulation capabilities and improve therapy efficacy while reducing the setup burden during the implant, or patient follow up visits.

When compared to other systems, the clinician and or technical support representative may spend time with the patient and a clinician programmer having the patient move to various postures states, e.g., reclining to standing, and calibrate the medical device parameters for each position. However, manually identifying patient thresholds, such as a perception threshold, can be time consuming and rely on subjective feedback from the patient. Therefore, clinicians may be pressed for time when setting up sensing and stimulation parameters, and patients may need to return to a clinic in order to update the medical device programming. Thus, manual setup may be frustrating for both the clinician and the patient. However, for the system of this disclosure, the generalized pre-calibrated positions may be enough to allow the system to automatically adjust one or more operating parameters of the set of operating parameters without extensive post implant calibration.

The processing circuitry of system 100 may monitor the sensor data, such as the accelerometer, gyroscopes, and similar sensors of sensors 222 (608) during the normal patient activity. The processing circuitry may dynamically and automatically switch simulation artifact cancellation parameters (e.g., filter coefficients), sensing parameters and/or stimulation parameters based on the detected physical state change (610).

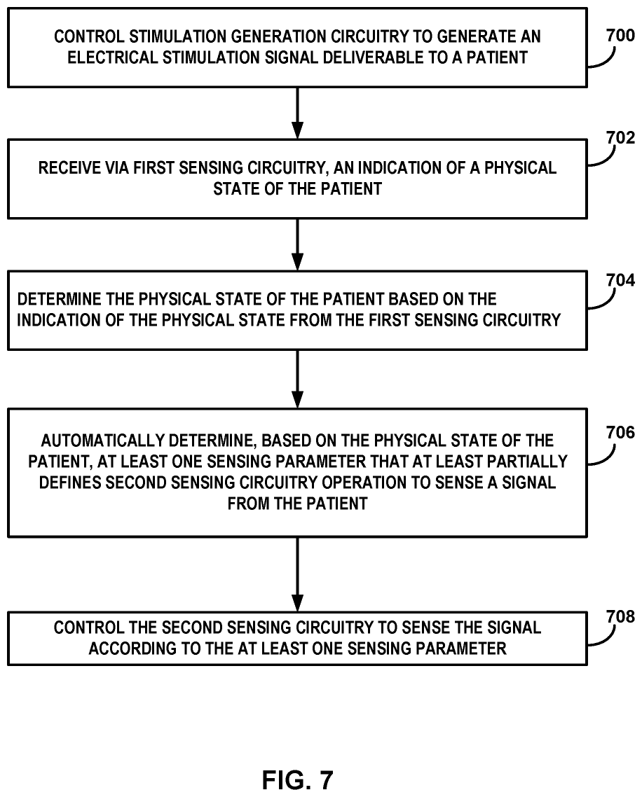

FIG. 7 is a flowchart illustrating an example operation of the medical system of this disclosure including automatically adjusting parameters based on patient state. The blocks of FIG. 7 may be performed, for example, by components of FIGS. 1, 2A and 2B.

Processing circuitry, e.g., processing circuitry 210 of IMD 200, processing circuitry 252 of computing device 290, or processing circuitry of servers 160, may control stimulation generation circuitry to generate an electrical stimulation signal deliverable to target tissue of a patient (700). In some examples, the stimulation generation circuitry may be directly connected to the processing circuitry, such in IMD 110 of FIG. 1 and as stimulation generation circuitry 202 of IMD 200 shown in FIG. 2A. In other examples, the processing circuitry may communicate with the IMD to control the stimulation generation circuitry.

The processing circuitry may receive via first sensing circuitry, an indication of a physical state of the patient (702), e.g., an accelerometer, MEMS gyroscope or similar sensor on the IMD (sensors 222), as well as a sensor on wearable device 152. As described above in relation to FIGS. 1-6, the physical state may include a posture state and activity state.

The processing circuitry may determine the physical state of the patient based on the indication of the physical state from the first sensing circuitry (704), and automatically determine, based on the physical state of the patient, at least one sensing parameter, e.g., stored at storage device 212 of FIG. 2A, or associated with processing circuitry 252 of FIG. 2B, that at least partially defines second sensing circuitry operation to sense an evoked response signal elicited by the stimulation generation circuitry (706). As described above, the sensing parameters may include electrode configuration, sensing window, gain settings, amplifier settings, the selected channel, e.g., LFP vs ECAP channel, sampling rate, and other sensing parameters. In some examples, the processing circuitry may also adjust post processing parameters, as described above in relation to FIGS. 2A and 2B.

The processing circuitry may further control the second sensing circuitry, e.g., sensing circuitry 206 of FIG. 2A, to sense the evoked response signal according to the at least one sensing parameter (708). As with the stimulation circuitry, in some examples, the processing is directly connected to the sensing circuitry and in other examples, the processing circuitry may be located in an external computing device and communicate with the sensing circuitry in the IMD.

The techniques of this disclosure may also be described in the following examples.

Example 1: A device comprising processing circuitry configured to: control stimulation generation circuitry to generate an electrical stimulation signal deliverable to a patient; receive, via first sensing circuitry, an indication of a physical state of the patient; determine the physical state of the patient based on the indication of the physical state from the first sensing circuitry; and automatically determine, based on the physical state of the patient, at least one sensing parameter that at least partially defines second sensing circuitry operation to sense a signal from the patient, and control the second sensing circuitry to sense the signal according to the at least one sensing parameter.

Example 2: The device of example 1, wherein the at least one sensing parameter comprises at least one of the following: a sensing electrode configuration from a plurality of electrodes, a gain setting, post processing parameters, sample rate, a sensing channel selection, or amplifier settings.

Example 3: The device of any of examples 1 and 2, wherein the at least one sensing parameter comprises artifact cancellation parameters comprising filter coefficients, an algorithm deployment, a recharge setting, a template for subtraction, or a sensing window.

Example 4: The device of any of examples 1 through 3, wherein the physical state comprises one or more of: walking, stationary, mobile, at rest, a fall, exercise, or asleep.

Example 5: The device of any of examples 1 through 4, wherein the signal sensed from the patient comprises a response signal elicited by the stimulation generation circuitry, and wherein the processing circuitry is further configured to: receive from the second sensing circuitry the sensed evoked response signal; and automatically adjust, based on both the sensed evoked response signal and the determined physical state of the patient, the at least one sensing parameter.

Example 6: The device of example 5, wherein the processing circuitry is further configured to: automatically adjust at least one stimulation parameter of the set of stimulation parameters based on both the sensed evoked response signal and the determined physical state of the patient; and control delivery of the electrical stimulation according to the adjusted value of the at least one stimulation parameter.

Example 7: The of any of examples 1 through 6, wherein the response signal elicited by the stimulation generation circuitry comprises at least one of: an evoked response, or a change in a bioelectrical signal as a result of output delivered by the stimulation generation circuitry.

Example 8: The device of any of examples 1 through 7, wherein the physical state comprises one of: upright, reclining, bent over, lying front, lying back, lying left, or lying right.

Example 9: The device of any of examples 1 through 8, wherein the set of stimulation parameters comprise at least one of an amplitude, interpulse interval, a pulse width, a pulse frequency, or a pulse shape.

Example 10: The device of any of examples 1 through 9, wherein the device is an implantable medical device (IMD) further comprising stimulation generation circuitry configured to generate a stimulation signal, according to a set of stimulation parameters, to be delivered to target anatomy of the patient via a stimulation electrode configuration from a plurality of electrodes; and the first sensing circuitry configured to measure a physical state of the patient, wherein the physical state of the patient comprises at least an activity state or a posture state; the second sensing circuitry configured to sense a response signal responsive to the stimulation signal according to the at least one sensing parameter, wherein the processing circuitry is communicatively coupled to the stimulation generation circuitry, the first sensing circuitry and the second sensing circuitry.

Example 11: The device of any of examples 1 through 10, wherein the processing circuitry is configured to, responsive determining the physical state of the patient, change operation of the device by at least one of: suspend delivery of the stimulation signal when the stimulation generation circuitry is delivering stimulation to target anatomy of the patient; or deliver the stimulation signal when the stimulation signal is suspended.

Example 12: The device of any of examples 1 through 11, wherein the indication of the physical state is a first indication of the physical state, and wherein the processing circuitry is configured to: receive a second indication of the physical state of the patient from a second device, and determine the physical state of the patient based on the first indication of the physical state from the first sensing circuitry and the second indication of the physical state from the second device.

Example 13: A method comprising controlling, by processing circuitry operatively coupled to a memory, stimulation generation circuitry to generate an electrical stimulation signal deliverable to a patient; receiving, by the processing circuitry and via first sensing circuitry, an indication of a physical state of the patient; determining the physical state of the patient based on the indication of the physical state from the first sensing circuitry; and automatically determining, based on the physical state of the patient, at least one sensing parameter that at least partially defines second sensing circuitry operation to sense a signal from the patient, and controlling, by the processing circuitry, the second sensing circuitry to sense the signal according to the at least one sensing parameter.

Example 14: The method of example 13, wherein the processing circuitry is a component of an implantable medical device (IMD), the device comprising stimulation generation circuitry configured to generate a stimulation signal, according to a set of stimulation parameters, to be delivered to target anatomy of the patient via a stimulation electrode configuration from a plurality of electrodes; and the first sensing circuitry configured to measure the physical state of the patient, wherein the physical state of the patient comprises at least an activity state and a posture state; the second sensing circuitry configured to sense the signal from the patient according to the at least one sensing parameter.

Example 15: The method of any of examples 13 and 14, wherein the signal sensed from the patient comprises a response signal elicited by the stimulation generation circuitry, the method further comprising receiving, by the processing circuitry, from the second sensing circuitry the sensed response signal; automatically adjusting, by the processing circuitry, based on both the sensed response signal and the determined physical state of the patient, the at least one sensing parameter.

Example 16: The method of example 15, further comprising automatically adjusting, by the processing circuitry, at least one stimulation parameter of the set of stimulation parameters based on both the sensed response signal and the determined physical state of the patient; and controlling, by the processing circuitry, delivery of the electrical stimulation according to the adjusted value of the at least one stimulation parameter.

Example 17: The method of any of examples 13 through 16, wherein determining the physical state of the patient comprises at least determining, by the processing circuitry, that the physical state has changed from a first physical state to a second physical state.

Example 18: The method of any of examples 13 through 17, wherein the at least one sensing parameter comprises at least one of the following: a sensing electrode configuration from a plurality of electrodes, a gain setting, post processing parameters, a sensing channel selection, amplifier settings or artifact cancellation parameters comprising filter coefficients, an algorithm deployment, a recharge setting, a template for subtraction, or a sensing window.

Example 19: A non-transitory computer-readable storage medium comprising instructions that, when executed, cause processing circuitry of a computing device to: control stimulation generation circuitry to generate an electrical stimulation signal deliverable to a patient; receive via first sensing circuitry, an indication of a physical state of the patient; determine the physical state of the patient based on the indication of the physical state from the first sensing circuitry; and automatically determine, based on the physical state of the patient, at least one sensing parameter that at least partially defines second sensing circuitry operation to sense a signal from the patient, and control the second sensing circuitry to sense the signal according to the at least one sensing parameter.

Example 20: The non-transitory computer-readable storage medium of example 19, wherein the processing circuitry is a component of an implantable medical device (IMD) comprising stimulation generation circuitry configured to generate a stimulation signal, according to a set of stimulation parameters, to be delivered to target anatomy of the patient via a stimulation electrode configuration from a plurality of electrodes; and the first sensing circuitry configured to measure the physical state of the patient, wherein the physical state of the patient comprises at least an activity state and a posture state; the second sensing circuitry configured to sense a response signal responsive to the stimulation signal according to the at least one sensing parameter.

In one or more examples, the functions described above may be implemented in hardware, software, firmware, or any combination thereof. For example, the various components of FIGS. 1 and 2, such as IMD 110, IMD 200, processing circuitry 210, servers 160 and external device 150 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor” and “processing circuitry,” as used herein, such as processing circuitry 210, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

1-15. (canceled)

16. A device comprising:
processing circuitry configured to:
control stimulation generation circuitry to generate an electrical stimulation signal deliverable to a patient;
receive, via first sensing circuitry, an indication of a physical state of the patient;
determine the physical state of the patient based on the indication of the physical state from the first sensing circuitry; and
automatically determine, based on the physical state of the patient, at least one sensing parameter that at least partially defines second sensing circuitry operation to sense a signal from the patient, and
control the second sensing circuitry to sense the signal according to the at least one sensing parameter.

17. The device of claim 16, wherein the at least one sensing parameter comprises at least one of the following: a sensing electrode configuration from a plurality of electrodes, a gain setting, post processing parameters, a sensing channel selection, a sample rate, or amplifier settings.

18. The device of claim 16, wherein at least one sensing parameter comprises artifact cancellation parameters comprising at least one of the following: filter coefficients, an algorithm deployment, a recharge setting, a template for subtraction, or a sensing window.

19. The device of claim 16, wherein the physical state comprises one or more of:
walking, stationary, mobile, at rest, a fall, exercise, or asleep.

20. The device of claim 16,
wherein the signal sensed from the patient comprises a response signal elicited by the stimulation generation circuitry; and
wherein the processing circuitry is further configured to:
receive from the second sensing circuitry the sense—response signal; and
automatically adjust, based on both the sense—response signal and the determined physical state of the patient, the at least one sensing parameter.

21. The device of claim 20, wherein the processing circuitry is further configured to:
automatically adjust at least one stimulation parameter of the set of stimulation parameters based on both the sensed response signal and the determined physical state of the patient; and
control delivery of the electrical stimulation according to the adjusted value of the at least one stimulation parameter.

22. The device of claim 20, wherein the response signal elicited by the stimulation generation circuitry comprises at least one of: an evoked response, or a change in a bioelectrical signal as a result of output delivered by the stimulation generation circuitry.

23. The device of claim 16, wherein the physical state comprises one of: upright, reclining, bent over, lying front, lying back, lying left, or lying right.

24. The device of claim 16, wherein the set of stimulation parameters comprise at least one of an amplitude, a pulse width, an interpulse interval, a pulse frequency, or a pulse shape.

25. The device of claim 16,
wherein the device is an implantable medical device (IMD) further comprising:
stimulation generation circuitry configured to generate a stimulation signal, according to a set of stimulation parameters, to be delivered to target anatomy of the patient via a stimulation electrode configuration from a plurality of electrodes; and
the first sensing circuitry configured to measure a physical state of the patient, wherein the physical state of the patient comprises at least an activity state or a posture state;
the second sensing circuitry configured to sense the response signal responsive to the stimulation signal according to the at least one sensing parameter,
wherein the processing circuitry is communicatively coupled to the stimulation generation circuitry, the first sensing circuitry and the second sensing circuitry.

26. The device of claim 16, wherein the processing circuitry is configured to, responsive determining the physical state of the patient, change operation of the device by at least one of:
suspend delivery of the stimulation signal when the stimulation generation circuitry is delivering stimulation to target anatomy of the patient; or
deliver the stimulation signal when the stimulation signal delivery was suspended.

27. The device of claim 16, wherein the indication of the physical state is a first indication of the physical state, and wherein the processing circuitry is configured to:
receive a second indication of the physical state of the patient from a second device, and
determine the physical state of the patient based on the first indication of the physical state from the first sensing circuitry and the second indication of the physical state from the second device.

28. A method comprising:
controlling, by processing circuitry operatively coupled to a memory, stimulation generation circuitry to generate an electrical stimulation signal deliverable to a patient;
receiving, by the processing circuitry and via first sensing circuitry, an indication of a physical state of the patient;
determining the physical state of the patient based on the indication of the physical state from the first sensing circuitry; and
automatically determining, based on the physical state of the patient, at least one sensing parameter that at least partially defines second sensing circuitry operation to sense a signal from the patient, and
controlling, by the processing circuitry, the second sensing circuitry to sense the signal according to the at least one sensing parameter.

29. The method of claim 28,
wherein the processing circuitry is a component of an implantable medical device (IMD), the device comprising:
stimulation generation circuitry configured to generate a stimulation signal, according to a set of stimulation parameters, to be delivered to target anatomy of the patient via a stimulation electrode configuration from a plurality of electrodes; and
the first sensing circuitry configured to measure the physical state of the patient, wherein the physical state of the patient comprises at least an activity state and a posture state;
the second sensing circuitry configured to sense the signal from the patient according to the at least one sensing parameter.

30. The method of claim 28, wherein the signal sensed from the patient comprises a response signal elicited by the stimulation generation circuitry, the method further comprising:
receiving, by the processing circuitry, from the second sensing circuitry the sensed response signal;
automatically adjusting, by the processing circuitry, based on both the sensed response signal and the determined physical state of the patient, the at least one sensing parameter.

31. The method of claim 30, further comprising:
automatically adjusting, by the processing circuitry, at least one stimulation parameter of the set of stimulation parameters based on both the sensed response signal and the determined physical state of the patient; and
controlling, by the processing circuitry, delivery of the electrical stimulation according to the adjusted value of at least one stimulation parameter.

32. The method of claim 28, wherein determining the physical state of the patient comprises at least determining, by the processing circuitry, that the physical state has changed from a first physical state to a second physical state.

33. The method of claim 28, wherein the at least one sensing parameter comprises at least one of the following: a sensing electrode configuration from a plurality of electrodes, a gain setting, post processing parameters, a sensing channel selection, amplifier settings or artifact cancellation parameters comprising at least one of the following: filter coefficients, an algorithm deployment, a recharge setting, a template for subtraction, or a sensing window.

34. A non-transitory computer-readable storage medium comprising instructions that, when executed, cause processing circuitry of a computing device to:
control stimulation generation circuitry to generate an electrical stimulation signal deliverable to a patient;
receive via first sensing circuitry, an indication of a physical state of the patient;
determine the physical state of the patient based on the indication of the physical state from the first sensing circuitry; and
automatically determine, based on the physical state of the patient, at least one sensing parameter that at least partially defines second sensing circuitry operation to sense a signal from the patient, and
control the second sensing circuitry to sense the signal according to the at least one sensing parameter.

35. The non-transitory computer-readable storage medium of claim 19, wherein the processing circuitry is a component of an implantable medical device (IMD), the device comprising:
stimulation generation circuitry configured to generate a stimulation signal, according to a set of stimulation parameters, to be delivered to target anatomy of the patient via a stimulation electrode configuration from a plurality of electrodes; and
the first sensing circuitry configured to measure the physical state of the patient, wherein the physical state of the patient comprises at least an activity state and a posture state;
the second sensing circuitry configured to sense a response signal responsive to the stimulation signal according to at least one sensing parameter.
